(19) United States
(12) Patent Application Publication
(10) Pub. No.: US 2006/0293557 A1
(43) Pub. Date: Dec. 28, 2006

(54) Methods and apparati for surgical navigation and visualization with microscope ("Micro Dex-Ray")

(75) Inventors: Zhu Chuanggui, Singapore (SG); Kusuma Agusanto, Singapore (SG)

Correspondence Address: KRAMER LEVIN NAFTALIS & FRANKEL LLP, INTELLECTUAL PROPERTY DEPARTMENT, 1177 AVENUE OF THE AMERICAS, NEW YORK, NY, 10036, US

(73) Assignee: Bracco Imaging, s.p.a., Milano (IT)

(21) Appl. No.: 11/375,656

(22) Filed: Mar. 13, 2006

Related U.S. Application Data
(60) Provisional application No. 60/660,845, filed on Mar. 11, 2005.

(30) Foreign Application Priority Data
Jul. 20, 2005 (WO) PCT/SG05/00244

(51) Int. Cl. A61B 1/00

(52) U.S. Cl. 600/101

(57) ABSTRACT

An improved system and method for macroscopic and microscopic surgical navigation and visualization are presented. In exemplary embodiments of the present invention an integrated system can include a computer which has stored three dimensional representations of a patient's internal anatomy, a display, a probe and an operation microscope. In exemplary embodiments of the present invention reference markers can be attached to the probe and the microscope, and the system can also include a tracking system which can track the 3D position and orientation of each of the probe and microscope. In exemplary embodiments of the present invention a system can include means for detecting changes in the imaging parameters of the microscope, such as, for example, magnification and focus, which occur as a result of user adjustment and operation of the microscope. The microscope can have, for example, a focal point position relative to the markers attached to the microscope and can, for example, be calibrated in the full range of microscope focus. In exemplary embodiments of the present invention, the position of the microscope can be obtained from the tracking data regarding the microscope and the focus can be obtained from, for example, a sensor integrated with the microscope. Additionally, a tip position of the probe can also be obtained from the tracking data of the reference markers on the probe, and means can be provided for registration of virtual representations of patient anatomical data with real images from one or more cameras on each of the probe and the microscope. In exemplary embodiments of the present invention visualization and navigation can be provided by each of the microscope and the probe, and when both are active the system can intelligently display a microscopic or a macroscopic (probe based) augmented image according to defined rules.

### CROSS REFERENCE TO RELATED APPLICATIONS

This application claims the benefit of U.S. Provisional Patent Application No. 60/660,845, filed on Mar. 11, 2005, under common assignment herewith, which is hereby incorporated herein by this reference. This application also claims the benefit of PCT/SG2005/00244, entitled Systems and Methods For Mapping A Virtual Model Of An Object To The Object (“Multipoint Registration”) filed on 20 Jul. 2005, which is also incorporated herein by reference. This application also incorporates herein by reference the disclosure of U.S. patent application Ser. No. 10,832,902, filed on Apr. 27, 2004, and published as US Published Patent Application Publication No. 20050015005 (“the Camera-probe Application”).

### TECHNICAL FIELD

The present invention relates to image-based surgical guidance and visualization systems.

### BACKGROUND OF THE INVENTION

Neurosurgery is routinely conducted in two operational modes: a macroscopic mode and a microscopic mode. In the former a surgical field is generally viewed with the naked eye, and in the latter the surgical field is viewed through a microscope. In each of these operational modes, image based navigation and visualization systems have been used with success in aiding physicians to perform a wide variety of delicate surgical procedures.

In image based navigation and visualization, images depicting the internal anatomies of a patient are generated, usually from magnetic resonance imaging (MRI), computer tomography (CT), and a variety of other technologies, prior to or during a surgery. A three-dimensional (3D) representation of the patient is generated from the images. The representation can be in varies forms, from volume images and 3D models of varies anatomical structures of the patient reconstructed from the images, to drawings, annotations and measurements added to illustrate a surgical plan, and a combine of them. At surgery, the 3D representation is aligned with the patient by registration. By linking the images of internal anatomy with the actual surgical field, navigation systems can improve the surgeon's ability to locate various anatomical features inside the patient in the operation.

In macroscopic navigation, a user (surgeon) holds a probe which is tracked by a tracking device. When such a probe is introduced into a surgical filed, the position of the probe tip represented as an icon is drawn on the view of the 3D representation of the patient. Navigation helps the surgeon to decide the entry point, to understand the anatomic structures toward the target, and to avoid critical structures along the surgical path.

US Published Patent Application Publication No. 20050015005 describes an improved navigation system where the probe includes a micro camera. This enables augmented reality enhanced navigation within a given operative field by viewing real-time images acquired by the micro-camera overlaid on the 3D representation of the patient.

During microscopic surgery, an operation microscope is often used to provide a magnification of the surgical field within which a surgeon is working. The microscope can be tracked for navigation purposes and its focal point can be usually shown in the 3D representation in place of the probe tip.

To avoid having to look away from a surgical scene to a monitor, “image injection” microscopes have been developed where the navigation view generated by the computer workstation is superimposed on the optical image of the microscope. Such a superposition requires that the image seen through the microscope and the superimposed image data conform geometrically.

Current image overlay in microscope-based navigation systems consists of two-dimensional contours superimposed onto an optical image plane. To get a three-dimensional impression a surgeon has to scroll through different image planes and mentally merge the injected contours into a three-dimensional model.

Such conventional techniques allow a surgeon to navigate in a surgical field in both macroscopic surgery as well as when performing microscopic surgery. However, they also have the following significant drawbacks.

First, it is not unusual that during microscopic surgery, a surgeon would want to switch between the microscope based and probe based navigation and visualization. To do this, a surgeon usually must move the microscope up and/or away from the surgical field and then move the navigation probe into the surgical field, seriously interrupting normal surgical flow.

Second, to enable a surgeon to perform delicate procedures on microstructures, such as, for example, nerves and vessels, magnification of a microscope is usually set at a high level during the operation.

While this high level magnification does allow for the visualization of such microstructures, it also often limits the field of view. As the virtual image which can then be superimposed would have the same magnification ratio, the display of virtual objects is also limited. This can lead to a situation in which the surgeon cannot unambiguously identify an area that he is viewing through the microscope with an actual place on the patient. It is simply too small an area that he can view. As well, the overlay image may also not provide much useful information because the anatomic structures around the area are outside the field of view and thus not visible. Furthermore, under such circumstances a surgeon cannot see 3D structures of anatomic interest around the surgical field from a different point of view.

Third, during microscopic surgery, it is generally desirable for a surgeon to be fully aware of all of the structures around the surgical field. In conventional systems navigation views are superimposed on the optical view of the microscope. While this has the advantage that a surgeon can see a navigational view without looking away from the microscope, it has the disadvantages that only limited information in the navigation view can be displayed, that the display may seriously block the optical view of the surgeon, and the image injection increases the cost of the system.

Accordingly, what is needed in the art is a surgical navigation and visualization method and system which reduces the need to move off of the magnified view of a surgical field for navigation during microscopic surgery.

What is further needed in the art is a surgical imaging method and system which can provide integrated augmented reality enhanced microscopic and macroscopic navigation and visualization, as well as the facility to seamlessly and efficiently switch between them.

### SUMMARY OF THE INVENTION

An improved system and method for macroscopic and microscopic surgical navigation and visualization are presented. In exemplary embodiments of the present invention an integrated system can include a computer which has stored three dimensional representations of a patient's internal anatomy, a display, a probe and an operation microscope. In exemplary embodiments of the present invention reference markers can be attached to the probe and the microscope, and the system can also include a tracking system which can track the 3D position and orientation of each of the probe and microscope. In exemplary embodiments of the present invention a system can include means for detecting changes in the imaging parameters of the microscope, such as, for example, magnification and focus, which occur as a result of user adjustment and operation of the microscope. The microscope can have, for example, a focal point position relative to the markers attached to the microscope and can, for example, be calibrated in the full range of microscope focus. In exemplary embodiments of the present invention, the position of the microscope can be obtained from the tracking data regarding the microscope and the focus can be obtained from, for example, a sensor integrated with the microscope. Additionally, a tip position of the probe can also be obtained from the tracking data of the reference markers on the probe, and means can be provided for registration of virtual representations of patient anatomical data with real images from one or more cameras on each of the probe and the microscope. In exemplary embodiments of the present invention visualization and navigation images can be provided by each of the microscope and the probe, and when both are active the system can intelligently display either a microscopic or a macroscopic (probe based) real, virtual or augmented image according to defined rules.

### BRIEF DESCRIPTION OF THE DRAWINGS

FIGS. 1A-1C illustrate digital zooming of an augmented reality image according to an exemplary embodiment of the present invention;

FIG. 1D depicts an exemplary navigation system according to an exemplary embodiment of the present invention;

FIG. 2 shows a schematic depiction of a real image of an exemplary patient head according to an exemplary embodiment of the present invention;

FIG. 3 shows a schematic depiction of a virtual image of a tumor and blood vessel according to an exemplary embodiment of the present invention;

FIG. 4 shows a schematic depiction of a combined (augmented reality) image according to an exemplary embodiment of the present invention;

FIG. 5 shows a schematic depiction of a magnified augmented reality view according to an exemplary embodiment of the present invention;

FIG. 6 shows a schematic depiction of a magnified microscopic view according to an exemplary embodiment of the present invention;

FIG. 7 shows a schematic depiction of digitally zoomed-out (magnified) microscopic view according to an exemplary embodiment of the present invention;

FIG. 8 shows schematic depiction of an exemplary navigational view from a probe according to an exemplary embodiment of the present invention;

FIG. 9 shows an exemplary navigational view from a surgical microscope according to an exemplary embodiment of the present invention;

FIG. 10 shows the exemplary view of FIG. 9 after digitally zooming-in according to an exemplary embodiment of the present invention; and

FIG. 11 shows an exemplary augmented reality navigational view from an exemplary probe according to an exemplary embodiment of the present invention.

It is noted that the patent or application file contains at least one drawing executed in color. Copies of this patent or patent application publication with color drawings will be provided by the U.S. Patent Office upon request and payment of the necessary fee.

### DETAILED DESCRIPTION OF THE INVENTION

In exemplary embodiments of the present invention, navigation and visualization in both macroscopic and microscopic surgery can be smoothly facilitated and integrated. Thus, in such exemplary embodiments, there is no need to move a surgical microscope off of, or away from, a surgical field for navigation or visualization during a microscopic surgery to implement macroscopic navigation or visualization. Further, in exemplary embodiments of the present invention an augmented reality enhanced navigation system can be provided which can, for example, provide both microscopic and macroscopic navigational information of three-dimensional (3D) anatomic structures of the patient to a surgeon without the need to move the microscope off of, or away from, as the case may be, the surgical field.

In exemplary embodiments of the present invention, a video camera can, for example, be rigidly attached to a microscope. A computer can, for example, store a virtual microscope camera model having the same imaging properties and pose (position and orientation) as the corresponding actual video camera, said imaging properties including focal length, field of view and distortion parameters, zoom and focus. In exemplary embodiments of the present invention means can be provided to generate an augmented view for microscopic navigation by overlaying the video images from the camera, or cameras, as the case may be, on the microscope with virtual rendered images of the patient's 3D anatomical structures generated by the computer according to the corresponding virtual microscope camera model in response to the position and orientation data of the microscope from the tracking device as well as magnification and focus data obtained form the microscope itself, by, for example, an integrated sensor.

In exemplary embodiments of the present invention there can also be a video camera integrated with a probe, such as, for example, is described in the Camera-probe Applicaiton. As described in the Camera-probe Application, a virtual model of the video camera having the same imaging properties and pose (position and orientation) as the actual video camera, said imaging properties including focal length, field of view and distortion parameters, can be provided. Further, means can be provided to generate an augmented view for macroscopic navigation by overlaying video images from the camera in the probe with rendered images of the patient's 3D anatomical structures generated by the computer according to the virtual camera model in response to the position and orientation data of the probe from the tracking device.

In exemplary embodiments of the present invention an augmented microscopic view can be digitally zoomed so that a magnified view of microscopic navigation can be obtained without requiring a change of the position and settings (magnification and focus) of the microscope. An anatomic structure outside of the optical field of the microscope at its current settings can thus be displayed in such a zoomed-out display, overlayed only partly by the real time video image coming from the microscope's camera in the center of the display. Additionally, in exemplary embodiments of the present invention a user need not change the setting of or move the microscope away to obtain a macroscopic navigation view. A user need only move the probe, which can image the surgical field form any arbitrary viewpoint.

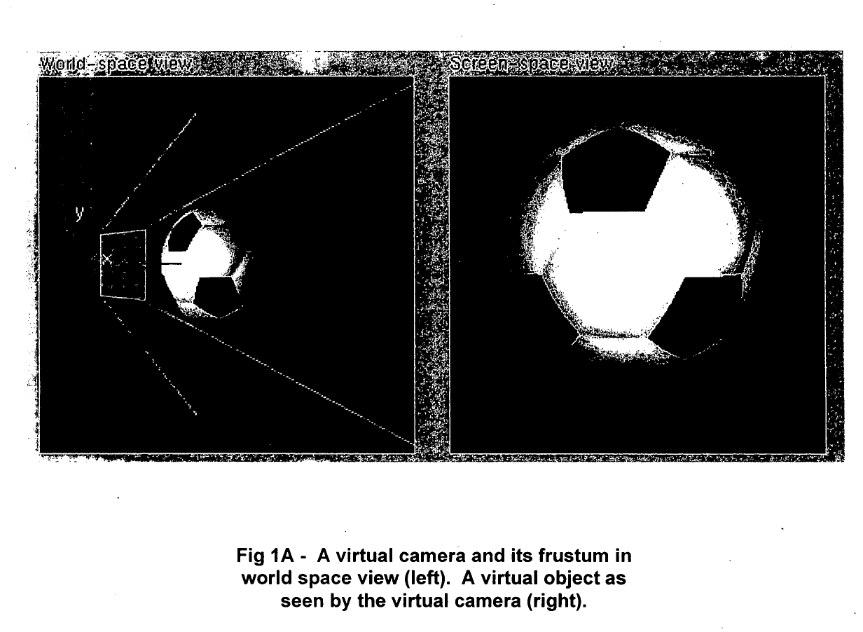

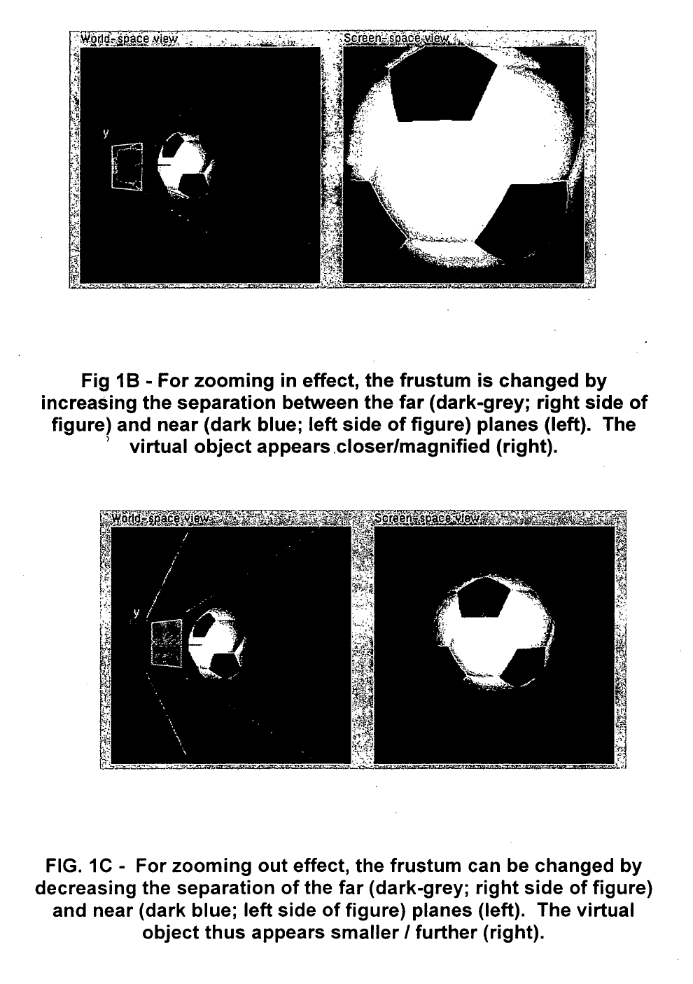

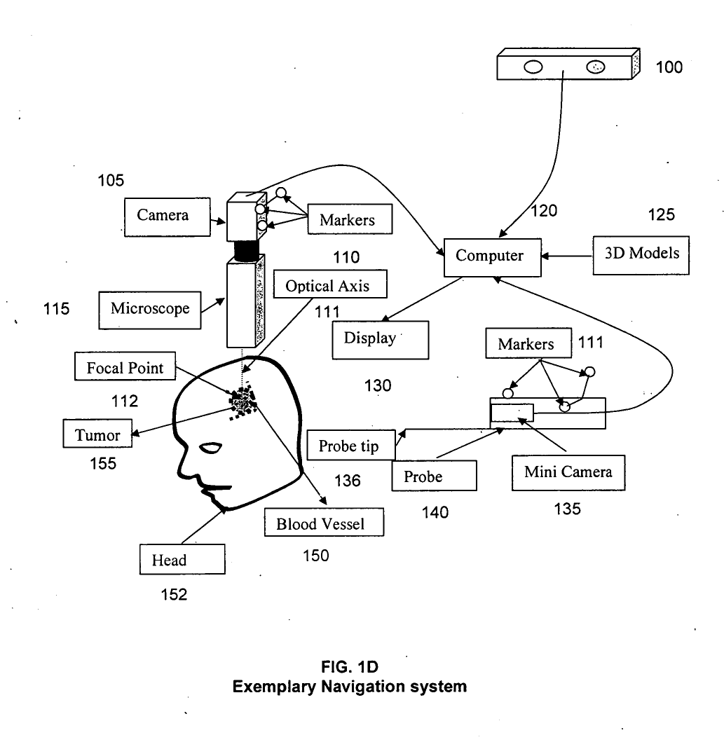

As noted above, the microscopic image can be digitally zoomed. This is next described. Change of magnification or zoom in an AR image operates by changing the field of view of a virtual camera (i.e. its frustum shape) together with the real image by insuring that the video image plane is aligned along the frustum of the virtual camera. This concept is illustrated with reference to FIGS. 1A-1C. It is noted that the original figures were in color, and the following description makes reference to those colors. However, the referents are easily discernable even in greyscale images.

FIG. 1A depicts a virtual camera (red axes at left of left image), and its frustum, represented by a near plane (dark blue; left side of left image) connected to a far plane (dark grey; right edge of left image), together with a virtual object.

The video image (pink rectangle), has its image centers aligned to the center of the frustum. In this setting, for example, the video image size is set to be the same as the near plane. Thus, the full video image covers the screen-view (or viewport), and there is no zooming effect.

In FIG. 1B the frustum has been changed such that a virtual object is projected with a magnification or zooming-in effect. Such change in frustum causes a change in what is visible in the screen space for the video image. Because now only some parts of the video image are inside the projection plane (near-plane), covering the screen view, there is a zooming-in effect also in the video image.

In FIG. 1C the frustum is changed such that the virtual object is projected with a zooming-out effect (appearing smaller). This change in frustum causes the whole video image inside the projection plane (near-plane) to cover only a part of the screen-view, thus the video image appears smaller in the screen view.

In exemplary embodiments of the present invention, a change of frustum can be achieved by changing the parameters of the perspective matrix of the virtual camera that produces the perspective projection. Specifically, for example, a perspective projection matrix of 4×4 matrix defined in an OpenGL context can, for example, be defined with the following parameters:
ProjMat [0]=2*Near/(Right−Left)* zoomFactor
ProjMat [2]=(Right+Left)/(Right−Left)
ProjMat [5]=2*Near/(Top−Bottom)*zoomFactor
ProjMat [6]=(Top+Bottom)/(Top−Bottom)
ProjMat [10]=−(Far+Near)/(Far−Near)
ProjMat [11]=−2*Far*Near/(Far−Near)
ProjMat [14]=−1
ProjMat [15]=0
with element 1,3,4,7,8,9,12, and 13 having value of 0 (read from left to right, top to bottom rule).

The parameters Left, Right, Top, and Bottom are functions of a microscope model based on intrinsic camera calibration parameters together with a focus and zoom setting of the microscope. The parameters for Near and Far can be, for example, set at constant values.

The parameter zoomFactor is the factor that can determine the zooming-in or zooming-out effects. When its value is below 1, for example, the effect is zooming-out, and when greater than 1, for example, the effect is zooming-in. No zoom effect is operative when the value is 1, for example.

In exemplary embodiments of the present invention, a video image can be displayed as a texture map with orthographic projection. To enable a correct and consistent overlay of a virtual object in the video image during zooming-in or zooming-out, an OpenGL viewport can be adjusted, for example, by the following parameters:
GLfloat cx=fabs (Left)/(Right−Left)
GLfloat cy=fabs(Bottom)/(Top−Bottom)
glviewport ((1−zoomFactor)*screenWidth*cx+originx, (1−zoomFactor)*screenHeight*cy+originY, screenWidth*zoomFactor screenHeight*zoomFactor);
which is, basically, scaling the size of screen view with a zoomFactor, and shifting the origin of the viewport according to the zoomFactor, video-image centers (cx, and cy), and the origin of OpenGL window such that the visible video image is overlayed correctly with the virtual image.

In exemplary embodiments of the present invention a probe can be used during microscopic surgery to obtain navigational views from varying orientations and locations. Anatomic structures around the surgical field, together with the focal points and optical axis of the microscope can, for example, be displayed from the point of view of the probe camera. The anatomic structures around the surgical area from various view points can, for example, thus be presented to the surgeon without the need of changing the microscope.

With reference to FIG. 1D, a surgical navigation system as used in performing a neurosurgical procedure according to an exemplary embodiment of the present invention is shown. In the figure the surgery is in the microscopic mode. Operation microscope 115 has a camera 105, which can, for example, be a color camera, installed on its imaging port and reference markers 110 can be mounted to it. The microscope 115 can, for example, have a built-in sensor to detect changes in imaging parameters of the microscope occurring as a result of adjustment of the microscope wherein said imaging parameters can include, for example, parameters comprising microscope magnification and focus. Such a sensor can be, for example, an encoder. The adjustment of focus and zoom involves mechanical movement of the lenses and such an encoder can, for example, measure such movement. The parameters can be available from a serial port of the microscope. The data format can be, for example, of the form Zoom: +120; Focus: 362. The microscope can also have an optical axis 111 and a focal point 112 which is defined as the intersection point of the optical axis and the focus plane of the microscope. A focus plane is perpendicular to the optical axis. On the focus plane the clearest image can, for example, be obtained. A focal plane can change with focus adjustment. In exemplary embodiments of the present invention a focal point's position relative to reference markers 110 can be calibrated in the full range of microscope focus and therefore can be obtained from the tracking data.

In FIG. 1D the microscope is being viewed by a surgeon and in the microscope's light path there is a patient's head 152. The exemplary patient has a tumor 155 (which is the target object of the operation) and a blood vessel structure 150 (which should be avoided during the operation) close to tumor 155. A position tracking system 100 (such as, for example, NDI Polaris) can receive commands from and can send tracking data to a computer 120, either, for example, wirelessly or through a cable linked with the computer, or using other known data transfer techniques.

Computer 120 can have 3D models 125 of the tumor 155 and blood vessel structure 150 stored in its memory prior to a navigation/visualization or other procedure according to an exemplary embodiment of the present invention. Such models can be stored, for example, after pre-operative scanning and processing of such scan data into a volumetric data set containing various segmentations and planning data. A probe 140 can, for example, contain a video camera 135, and a pointer with a tip 136 can be attached to its front end. The probe 140 can be placed within easy reach of a surgeon to facilitate its use during the surgery. The probe can, for example, be of the type as disclosed in the Camera-probe Application. The position tracking system 100 can, for example, provide continual real time tracking data of the microscope 115 to the computer. When the probe 140 is introduced into the surgical field, the position tracking system 100 can, for example, also provide continual real time tracking data of the probe 140 to the computer. The computer can be connected to (i) a display 130, (ii) a camera and sensor of microscope 115, and (iii) a mini camera of the probe. The system can, for example, further include software to detect position and orientation data of the microscope and probe from the tracking data, and from such position data to automatically select one (probe or microscope) to be used as a basis of images for navigation and/or visualization. Such automatic selection can be according to defined priority rules or various algorithms as may be appropriate to a given application and a given user's preferences.

For example, a given user may prefer to get his general bearings via a macroscopic view, and then when he gets close to delicate structures, use a microscopic view. If an operation has multiple stages, it can easily be seen that such a surgeon would cycle through using the probe, then the microscope, then again the probe and then again the microscope. For such a surgeon, the system could realize that for an initial period the main implement is a probe, and then once a microscope has been engaged it is the main implement until a new microscope position has been chosen, when the probe is once again used at the beginning of another stage. The system could, as a result, generate a combined image on the display corresponding to a view from whichever implement was then prioritized. Many alternative rules could be implemented, and a surgeon could always override such priority settings by actuating a switch or voice controlled or other known interface.

Continuing with reference to FIG. 1D, the computer 120 can, for example, receive a real-time video image of a surgical scene acquired by microscope camera 105. Microscope camera 105 can, for example, have a microscope virtual camera model which can be been provided and stored in computer 120.

In exemplary embodiments of the present invention a microscope virtual camera model can have a set of intrinsic parameters and extrinsic parameters wherein said intrinsic parameters can include, for example, focal length, image center and distortion, and said extrinsic parameters can include, for example, position and orientation of the virtual microscope camera model in relative to a reference coordinate system.

In exemplary embodiments of to the present invention a reference coordinate system can be, for example, the coordinate system of markers 110 which are rigidly linked to microscope 115.

In exemplary embodiments of the present invention the intrinsic and extrinsic parameters of the microscope camera model can change according to changes of the microscope's magnification and focus.

In exemplary embodiments according to the present invention the intrinsic and extrinsic parameters of a microscope camera model can, for example, be described as bivariate polynomial functions of the microscope magnification and focus. For example, a parameter ρ (ρ represents one of the intrinsic and extrinsic parameters) can be modeled as a qth order bivariate polynomial function of the values of focus (f) and zoom (z) of the microscope, for example, as follows:

$$ρ ( z , f ) = {\sum }_{m , n} {a}_{m , n} {z}^{m} {f}^{n} \\ ( m , n \geq 0 ; m + n \leq q ) .$$

To solve for coefficients a_{m,n,} the microscope can be calibrated as a number of fixed cameras (with fixed focal length) across the full range of the microscope focus and zoom range. After a sufficient number of fixed camera calibrations, under different zoom and focus settings, a group of calibration data can be obtained. The coefficients a_{m,n} of the polynomial functions can then be solved, for example, by bivariate polynomial fitting.

An exemplary microscope camera model for an exemplary microscope in an augmented reality microscope system can be expressed as follows:

Intrinsic Parameters

Image Size: Nx=768, Ny=576

Image Center: Cx=384, Cy=288

Focal Length:
fx=−0.000000008*F*Zˆ3+(−0.000004613)*F*Zˆ2+(−0.001289058*F*Z+(−0.022283345)*F+0.000039765*Zˆ3+0.042230380*Zˆ2+21.010557606*Z+4970.548674307
fy=0.000000010*F*Zˆ3+(−0.000001564)*F*Zˆ2+(−0.001287695)*F*Z+(−0.020680795)*F+0.000034475*Zˆ3+0.040391899Zˆ2+20.227847227*Z+4767.037899857

Extrinsic Parameters
Owcx=0.000008797*F+(−0.058476064)
Owcy=−0.000016119*F+(−0.781894036)
Owcz=−0.000004200*F+(−0.078145268)
Twcx=0.000000000*Fˆ2*Z+(−0.000000747)*Fˆ2+(−0.000002558)*F*Z+(−0.006475870)*F+0.000141871*Z+0.271534556
Twcy=−0.000000001*Fˆ2*Z+(−0.000001826)*Fˆ2+(0.000002707)*F*Z+(−0.004741056)*F+(−0.003616348)*Z+5.606256436
Twcz=0.000000302*Fˆ2*Z+0.000014187*Fˆ2+(−0.000088499)*F*Z+(−0.018100412)*F+0.061825291*Z+422.480480324.

In the above expression, Owcx, Owcy, Owcz are rotation vectors from which the rotation matrix from the microscope camera to the reference coordinate system can be calculated, and Twcx, Twcy, and Twcz are transforms in x, y and z and from them the transform matrix to the reference coordinate system can be constructed.

Thus, in exemplary embodiments of the present invention, for any given zoom and focus value of the microscope, a corresponding virtual microscope camera can be created and can be used to generate a virtual image of the virtual objects.

As is illustrated in FIG. 1D, computer 120 can receive the current magnification and focus values for the microscope. Intrinsic and extrinsic parameters of a virtual microscope camera can thus be calculated from the stored microscope camera model. The virtual microscope camera position and orientation in the position tracking system can be depicted using the tracking data of the markers on the microscope.

As is illustrated in FIG. 1D, the microscope has an optical axis 111 and a focal point 112. In exemplary embodiments according to the present invention the position of the focal point changes relative to the reference markers according to the changes of the microscope focus.

In exemplary embodiments according to the present invention the position of the focal point of the microscope relative to the reference markers can be calibrated before navigation. An exemplary calibrated result of the focal point for an exemplary microscope from an augmented reality microscope system is presented below.

FocusPoint (x, y, z)=(Fpx, Fpy, Fpz), wherein
Fpx=−0.000001113*Fˆ2+0.001109120*F+116.090108990;
Fpy=0.000002183*Fˆ2+(−0.000711078)*F+(−27.066366422);
Fpx=−0.000073468*Fˆ2+(−0.154217215)*F+−369.813473763; and

F represents focus.

A calibration result of the focal point can, for example, be stored in the computer. Thus, for any given focus value of the microscope, a position of focal point can be obtained from the tracking data of the reference markers.

In exemplary embodiments according to the present invention the optical axis can be, for example, a line linking the focal points of various microscope focal values.

In exemplary embodiments of the present invention image data of a patient can be mapped to the patient using one of the generally known registration techniques. For example, one such registration technique maps the image data of a patient to the patient using a number of anatomical features (at least three) on the body surface of the patient by matching their positions identified and located in the scan images and their corresponding positions on the patient determined using a tracked probe. The registration accuracy may be further improved by mapping a surface of a body part of the patient generated from the imaging data to the surface data of the corresponding body part generated on the operating table. For example, this method is described in detail in PCT/SG2005/00244, entitled “Systems and Methods For Mapping A Virtual Model Of An Object To The Object (“Multipoint Registration”)” filed on 20 Jul. 2005 by Applicant hereof. The registration method described in this PCT application can be used directly for microscope navigation in exemplary embodiments hereof. The aim of registration is to make the patient imaging data align with the patient, and it can be done, for example, in a macroscopic stage when the microscope is not involved yet, and the registration result used in microscopic navigation. After registration, the image data of the patient, including all the segmented objects and other objects generated in surgical planning associated with the imaging data, are registered to the physical patient. For example, in FIG. 1D the model of the tumor and blood vessel stored in computer 120 are registered with the actual tumor 155 and blood vessel 150 in the head of the patient.

The position and orientation of the patient head 152 and the position and orientation of the microscope video camera 105 can be transformed into a common coordinate system, for example the coordinate system of the position tracking system. The relative position and orientation between the head 152 and the microscope video camera 105 can thus be determined dynamically using the position tracking system 100.

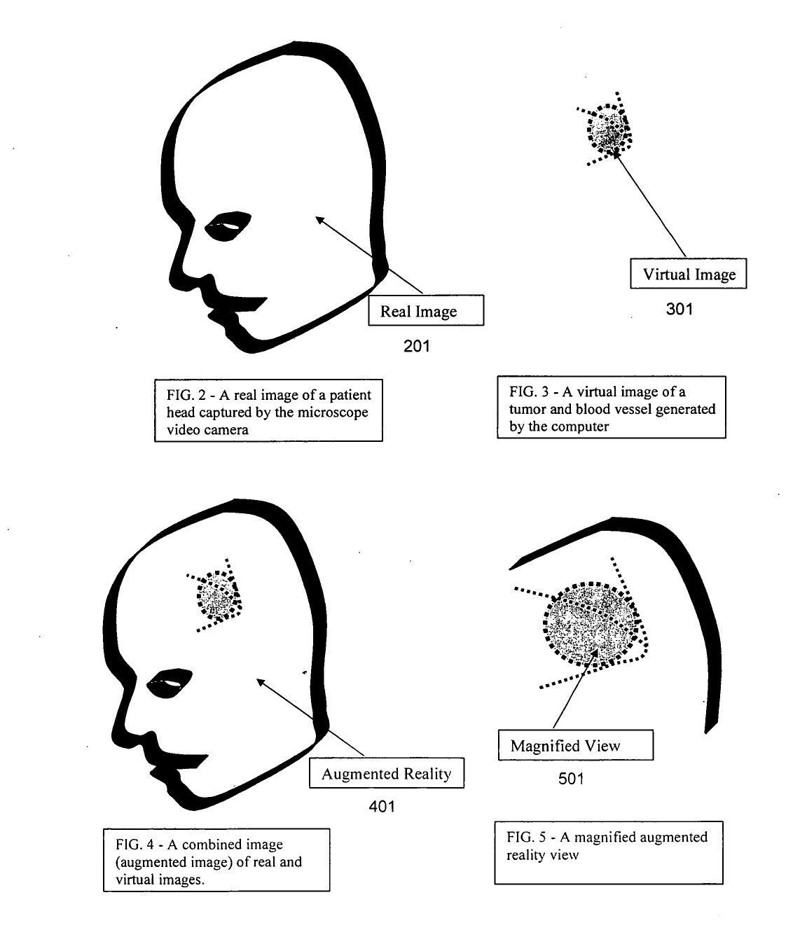

As is illustrated in FIG. 2, in exemplary embodiments of the present invention the microscope camera can capture a video image of patient head 152. The tumor 155 and blood vessel 150 may not be visible in the video image (as they may be visually occluded by an as yet closed portion of the head).

As illustrated In FIG. 3, in exemplary embodiments of the present invention the computer can generate a virtual image of tumor 155 and blood vessel 150 based on the intrinsic and extrinsic parameters of the virtual microscope camera and the stored model of the tumor and blood vessel.

As is illustrated in FIG. 4, in exemplary embodiments of the present invention real image 201 and virtual image 301 can be combined to generate an augmented reality image. The augmented reality image can then, for example, be on display device 130. Display 130 can be a monitor, a HMD, a display build in the microscope for “image injection”, etc.

The 3D model of tumor and blood vessel can be, for example, generated from three-dimensional (3D) images of a patient. For example, from MRI or CT images of the patient head. In exemplary embodiments of the present invention, such data can be generated using hardware and software provided by Volume Interactions Pte Ltd., such as, for example, the Dextroscope™ system running RadioDexter™ software.

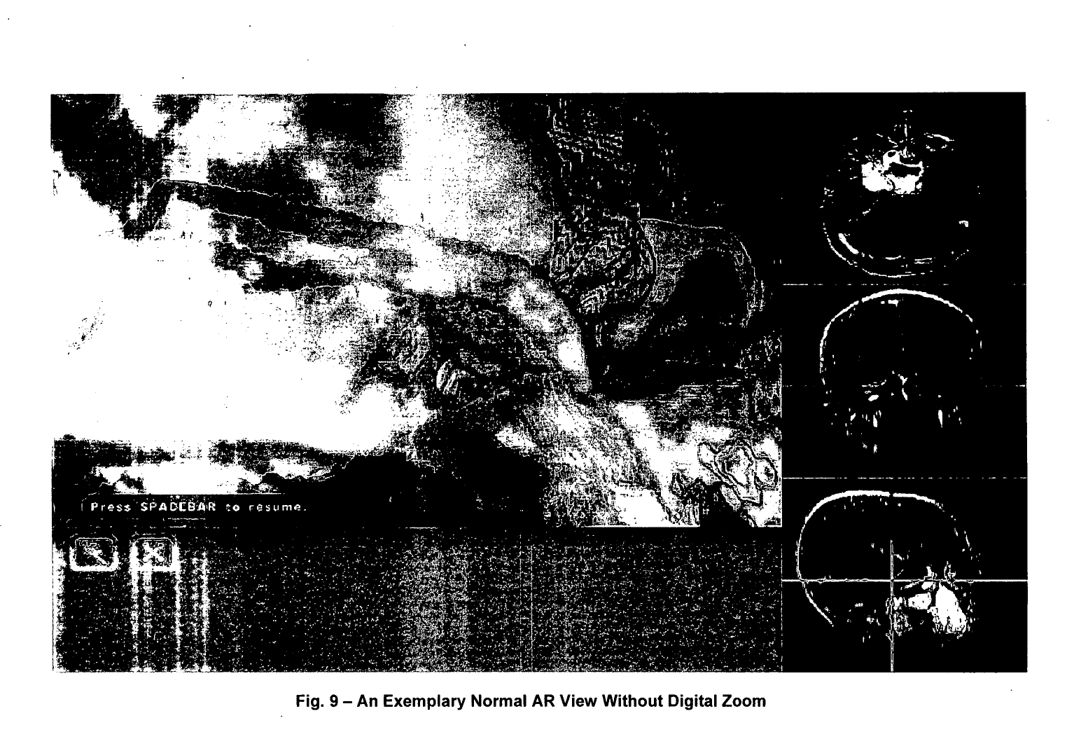

In exemplary embodiments according to the present invention the augmented reality image can be displayed in various ways. The real image can be overlaid on the virtual image (real image is on the virtual image), or be overlaid by the virtual image (the virtual image is on the real image). The transparency of the overlay image can be changed so that the augmented reality image can be displayed in various ways, with the virtual image only, real image only, or a combined view. At the same time, for example, axial, coronal and sagittal planes of the 3D models according to the position changing of the focal point can be displayed in three separate windows, as is shown, for example, in FIGS. 9-11.

In exemplary embodiments according to the present invention the augmented reality in microscopic navigation can be in various microscope settings across the full magnification and focus range.

FIG. 5 shows an exemplary augmented reality view of the patient head in a different (greater, relative to FIGS. 3-4) magnification setting.

In exemplary embodiments according to the present invention digital zoom can be used to virtually change the magnification of the augmented reality image. The zoom ratio can be an input of a user. The zoomed field of view can, for example, be centered at the center of the window by default.

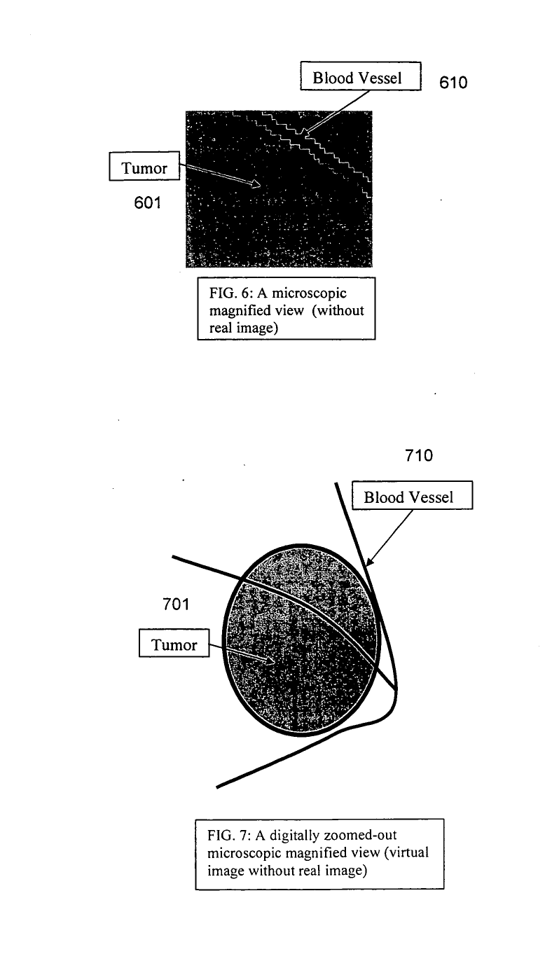

FIG. 6 shows an exemplary virtual image only navigation view of the surgical field through the microscope at a higher magnification. In this example, a surgeon is operating on the tumor so part of the tumor is visible in the optical view of the microscope. However, most of the tumor and all of the blood vessel are either hidden under the exposed surface or out of the field of view of the microscope so that the surgeon cannot see directly. A rendered image of tumor and blood vessel generated by the computer can be displayed to the surgeon, but because of the magnification, only a small part of the tumor and blood vessel can be shown.

In many contexts it can be crucial to know the exact 3D structure and location of the tumor and blood vessel beyond the field of view of the microscope without changing the microscope magnification and position. Thus, for example, FIG. 7 shows a virtually enlarged view of the microscope in which the whole structure of the tumor and blood vessel are visible. In exemplary embodiments of the present invention this can be achieved by digital zooming. Digital zooming virtually changes the field of view of the virtual microscope camera model, so that the 3D models in the virtual camera's field of view can be rendered from the same viewpoint but a different field of view. Digital zooming enables the surgeon to see beyond of the microscope's field of view without changing the microscope's actual settings. In exemplary embodiments of the present invention the video signal can also be zoomed, and thus a zoomed image can have video (real) images, virtual images or any combination of both, with varying transparency of either. FIG. 7 is zoomed-out relative to the view of FIG. 6, but obviously of a much greater magnification (zoom-in) relative to the view of FIG. 5 and of course relative to that of FIG. 3. Thus, a user may frequently change zoom values, zooming in and out repeatedly over the course of a given procedure or operation.

In a neurosurgical application scenario, a surgeon may, for example, use the probe 140 to do registration, and to select the entrance point by navigating with the probe. Then, for example, the microscope can be brought in for refined navigation and guidance. During surgery, a surgeon may need from time to time to navigate using the probe 140, as navigation by moving the probe 140 can be easier to handle than navigation by moving the microscope. In such an exemplary application scenario, an exemplary system can allow for swift and smooth shift between the two navigation methods.

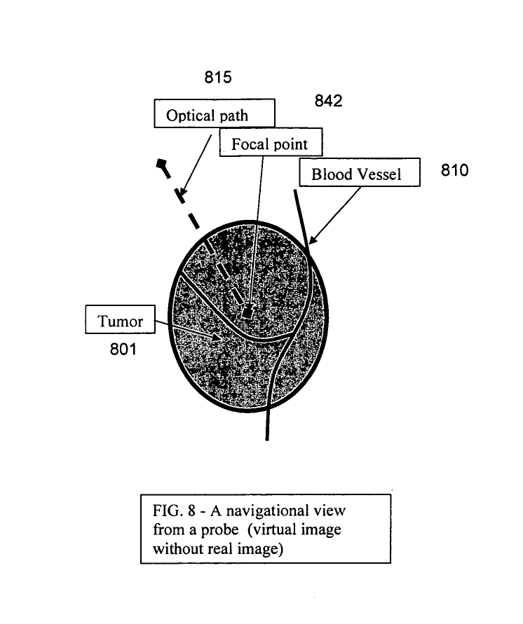

FIG. 8 depicts the exemplary scene of FIG. 7 from the point of view of the mini-camera inside the probe. The focal point as well as the optical path of the microscope can, for example, be shown together with the tumor and blood vessels, indicating the 3D relationship of the microscope, the surgical field and the virtual objects (e.g., tumor and blood vessels).

FIGS. 9-11 are actual screen shots from an exemplary embodiment of the present invention. FIG. 9 shows an exemplary navigational view from a surgical microscope according to an exemplary embodiment of the present invention.

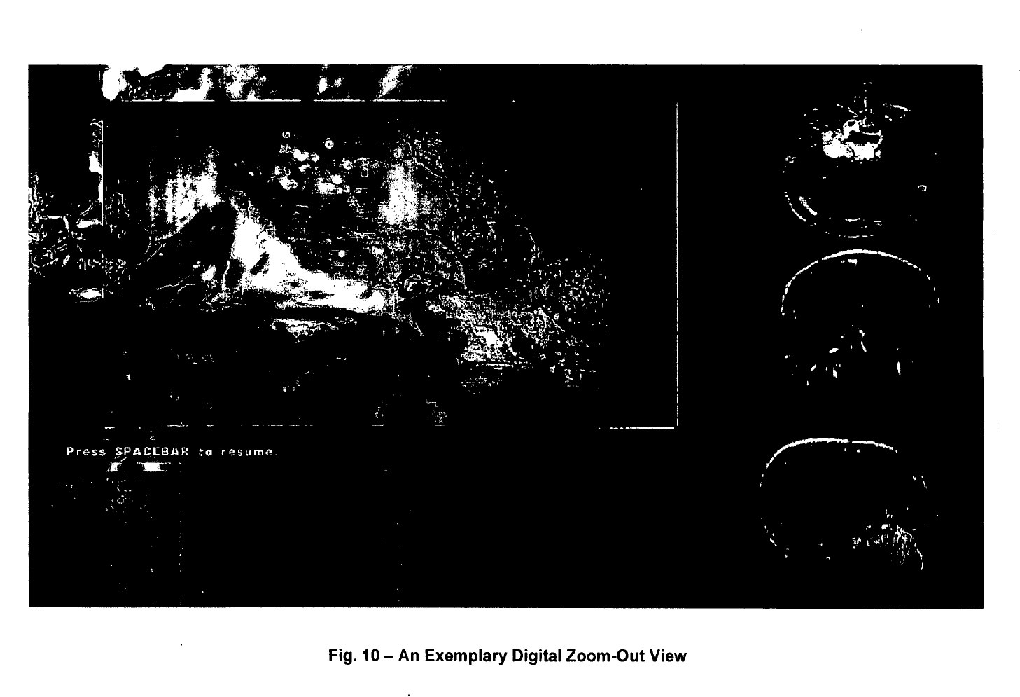

FIG. 10 shows the exemplary view of FIG. 9 after digitally zooming-out according to an exemplary embodiment of the present invention, using the techniques described above as in connection with FIG. 7. Thus, FIG. 10, illustrates the difference between video and real images. A virtual image can, for example, always be larger than the video image, and this allows a user to see what is extending outside of or beyond the video window, and interpret it as a virtual object.

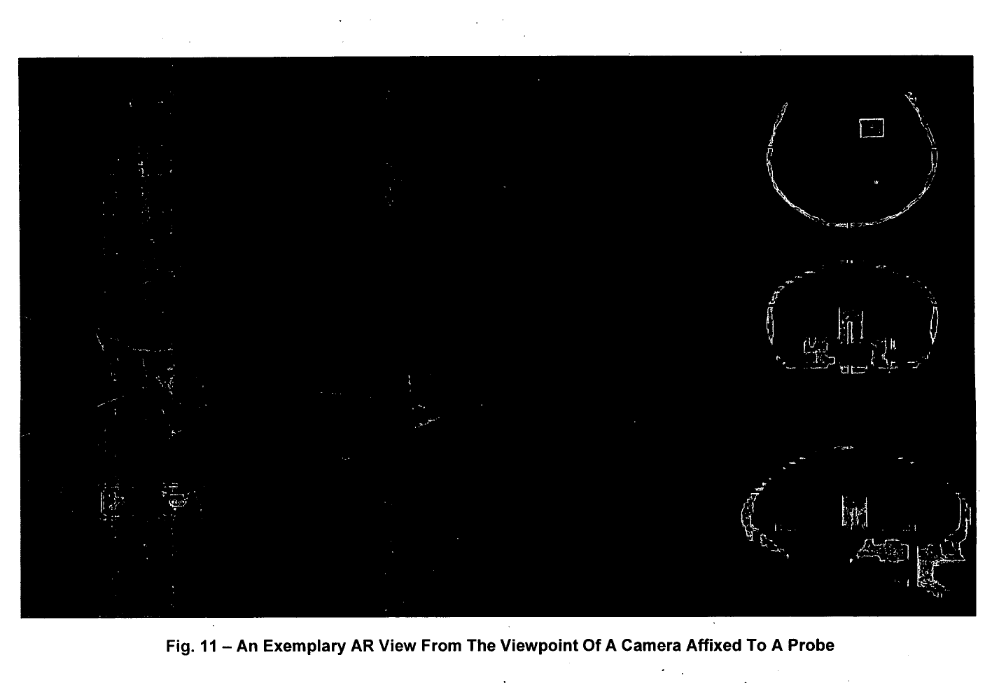

FIG. 11 shows an exemplary augmented reality navigational view from an exemplary probe according to an exemplary embodiment of the present invention, corresponding somewhat to that shown in FIG. 8, with the green dotted line at the left of the image represents the optical path and the cross hair underneath it (at approximately the center of the top surface of the yellow cylinder) represents the focal point of the microscope.

In exemplary embodiments according to the present invention the selection between the microscope and probe can be performed automatically. The automatic selection can be based upon (i.e., be a function of) the tracking data. In exemplary embodiments according to the present invention this can be achieved by setting a higher priority to the probe. If only the microscope tracking data is available, the microscope can, for example, be selected as the navigation instrument and its AR image can be displayed. If both the microscope and the probe are tracked, the probe can, for example, be selected and its AR view can be displayed. The microscope in such situation can, for example, be ignored. When the probe is not tracked, the microscope can, for example, be selected automatically for navigation. The video image can also be automatically changed accordingly.

Alternatively, other priority paradigms or algorithms can be implemented depending upon user preferences or the application or procedure an exemplary system is being used for. Thus which navigational tool's view is displayed, either microscope, or probe, can be dynamically modified as may be beneficial or useful. In any such priority algorithm a user can override a programmed priority via an interface. In exemplary embodiments of the present invention such interface can be acoustic (as in speaking a command or commands), visual, as by manipulating the probe in a defined space in a defined manner as described, for example, in the Camera-probe Application, tactile, such as, for example, via a footswitch, or other interface as may be known.

Notwithstanding the fact that in exemplary embodiments of the present invention either the probe based image/viewpoint or the microscope based image/viewpoint can be selected for display, in exemplary embodiments of the present invention both image feeds can be stored in a computer or memory device for later replay. Because once a real image viewpoint is known any virtual image which can be generated can be co-registered with it and displayed, storing all real video feeds, from both probe and microscope, with the respective positions and orientations of these devices, allows for the generation of any associated augmented reality at any subsequent time. This can allow for a “post mortem” of a given user's use of an exemplary system, for analysis of a user's skill, for learning which priority algorithm fits which user or application, for asking the “what if he visualized using the probe here as opposed to the microscope” type question, and for various other purposes.

The systems, methods and apparati of the present invention can thus enable a user to see “beyond the normal field of view” both during macroscopic surgery as well as during microscopic surgery. This allows a user to always be aware just how near he or she is to highly sensitive or important hidden structures, and to visualize anatomical structures and surgical pathways in an efficient and dynamic manner as may best be performed during various stages of a given procedure in a fully integrated, facile and responsive manner.

What is claimed:

1. An integrated surgical navigation and visualization system, comprising:
a microscope;
at least one video camera affixed to the microscope;
a computer;
a microscope camera model stored in the computer;
a probe;
a video camera affixed to the probe;
a probe camera model stored in the computer;
a tracking device arranged to determine poses of the probe and the microscope;
three dimensional patient image data stored in the computer; and
a display;
wherein in operation the computer automatically selects combined image data associated with either the probe or the microscope for display.

2. The system of claim 1, wherein said automatic selection is based on the tracking data and a defined relative priority algorithm of the probe view and the microscopic view.

3. The system of claim 3, wherein the microscope's magnification and focus are adjustable, and wherein a sensor detects the values of the magnification and focus and communicates this data to the computer.

4. The system of claim 1, wherein a virtual microscope camera which has imaging properties, position and orientation matching those of the video camera affixed to the microscope is generated from the microscope camera model, the microscope tracking data, and the microscope zoom and focus values.

5. The system of claim 1, wherein a position of the microscope's focal point in relation to a patient can be determined from the microscope's focus value and tracking data.

6. The system of claim 4, wherein the video image from the video camera affixed to the microscope is augmented by a virtual image generated by the computer from the three dimensional patient image data and a composite image is displayed on the display.

7. The system of claim 1, wherein a virtual probe camera having imaging properties, position and orientation matching those of the video camera affixed to the probe can be generated from the probe camera model and the probe tracking data.

8. The system of claim 4, wherein the video image from the video camera affixed to the probe is augmented by a virtual image generated by the computer from the three dimensional patient image data according to the virtual probe camera and a composite image is displayed on the display.

9. The system of claim 2, wherein the selection can be overridden by a user by actuating at least one of a visual, tactile, acoustic, or other interface.

10. A method of surgical navigation and visualization, comprising:
acquiring three dimensional image data from a patient;
storing said three dimensional image data;
registering the three dimensional image data to the patient;
acquiring real-time video images of the patient from a video camera affixed to a microscope;
tracking the position and orientation of the microscope;
receiving zoom and focus values of the microscope;
constructing a virtual microscope camera according to a microscope camera model, the tracking data, zoom and focus value;
generating a virtual image of a portion of the patient;
generating an augmented reality view by superimposing the real-time video images upon the virtual image; and
displaying said augmented reality view on one or more displays.

11. The method of claim 10, wherein the augmented reality view can be digitally zoomed without changing the position, zoom or focus value(s) of the microscope.

12. The method of claim 11, wherein the real image and virtual image are geometrically co-aligned in the digitally zoomed augmented reality view.

13. The method of claim 12, wherein the augmented reality view is zoomed out, the virtual image of three dimensional image data of the patient outside the field of view of the real image is generated and displayed as partially overlaid on the real image from the video camera.

14. The method of claim 13, further comprising automatically selecting a probe with an affixed video camera as an alternate navigational and visualization implement.

15. The method of claim 14, further comprising:
acquiring real-time video images of the patient from the video camera affixed to the probe;
tracking the position and orientation of the probe;
constructing a virtual probe camera according to a probe camera model and the tracking data;
generating a virtual image of three dimensional image data of the patient according to the virtual probe camera; and
generating an augmented reality view by superimposing the real-time video images from the probe upon said virtual image according to said virtual probe camera.

16. The method of claim 15, wherein the augmented reality view can be digitally zoomed without changing the position of the probe.

17. The method of claim 16, further comprising:
acquiring a real time video image of the patient from the camera affixed to the probe with the microscope remaining at the surgical operation condition;
generating a virtual image of the focal point and optical axis and the three dimensional image data of the patient according to the virtual probe camera; and
generating an augmented reality view by superimposing the real-time video images upon the virtual image.

18. The method of claim 10, including positioning the probe during microscopic surgery to obtain navigational views from varying orientations and locations.

19. The method of claim 18, wherein the anatomic structures around the surgical field, together with the focal points and optical axis of the microscope, can be displayed from the point of view of the probe camera on a display.

20. The method of claim 10, wherein the display is one of a monitor, a HMD, and a display built in the microscope for image injection.

21. The system of claim 1, wherein the display is one of a monitor, a HMD, and a display built in the microscope for image injection.
